(19) United States
(12) Patent Application Publication
(10) Pub. No.: US 2002/0127717 A1
(43) Pub. Date: Sep. 12, 2002

(54) Preparation of replicating macrophages and use in diagnosis and therapy

(76) Inventor: Syed Z. Salahuddin, Ventura, CA (US)

Correspondence Address: Barry S. Wilson, FOLEY & LARDNER, P.O. Box 80278, San Diego, CA, 92138-0278, US

(21) Appl. No.: 09/991,583

(22) Filed: Nov. 16, 2001

Related U.S. Application Data
(60) Provisional application No. 60/249,762, filed on Nov. 17, 2000.

(51) Int. Cl. C12N 5/08

(52) U.S. Cl. 435/372; 435/368

(57) ABSTRACT

Provided are methods for replicationg macrophages in vitro and cell culture composition comprising relicating macrophages. Also provided are methods for preparing replicating Kupffer cells from human liver. Further provided are methods for enhancing or extending immune function in an individual suffering from a deficiency relating to a reduced number of functional macrophages in a tissue or organ by administering a therapeutically effective amount of a replicating macrophages obtained by the methods of the invention.

### FIELD OF THE INVENTION

The present invention relates to the preparation and use of replicating macrophages including replicating human Kupffer cells in vitro.

### BACKGROUND OF THE INVENTION

Animals, including mammals, avians and reptiles have a variety of phagocytic cells or macrophages both circulating and fixed in tissues which are a part of the immune system. Macrophages contribute to the innate immune system as well as the acquired immune system, the latter involving presentation of foreign antigens to lymphocytes and elaboration of cytokine or lymphokine immune effector molecules. Such macrophages include blood monocytes, liver Kupffer cells, fixed tissue macrophages, dendritic cells including follicular dendritic cells of lymph nodes and spleen and skin dendritic cells also known as Langerhan's cells.

Kupffer's cells (KC) are large fixed macrophages which along with endothelial cells, make up the linings of the walls of the sinusoids of the liver. In humans, the liver is a vital organ, the second largest organ in the body, and very complex in function. Hepatocytes account for about 70% of the liver cell population and are involved in carbohydrate and protein and fat metabolism as well as detoxification. The liver is also an extramedulary hematopoitic organ that can produce all leukocyte lineages from resident hematopoitic stem cells. Despite its ability to regenerate and withstand abuse, the liver can be severely damaged by agents such as viruses, alcohol, drugs, trauma, and cancer cells. Among infectious agents, human hepatitis C virus (HCV) has emerged as one of the leading cause of catastrophic liver disease world wide in humans. Chronic hepatitis due to HCV is a commonly progressive viral disease and an important public health problem. Large percentages of chronic HCV infection can lead progressively to cirrhosis of liver and also to primary hepato-cellular carcinoma. KC play a major role in the physiological maintenance of hepatic architecture and wound healing process from chronic liver injury by removing soluble and particulate matter from the circulation.

In view of the important role played by macrophages in the body and particularly in the liver, there is a need for renewable sources of such cells that can be propagated in vitro. Such cells would be useful in transplantation such as a therapy for chronic liver disease and would provide a model system for investigation of liver viral infection and pathology.

### BRIEF SUMMARY OF THE INVENTION

In accordance with the present invention, provided are replicating macrophage compositions including replicating human Kupffer's cells and methods of making and using same.

In one aspect of the invention, there is provided a composition comprising a culture of replicating macrophages wherein at least some of the macrophages have undergone cell division during culture in vitro.

In another aspect of the invention, there is provided a method of culturing macrophages in vitro such that at least some of the macrophages have undergone cell division during culture. The method comprises growing the cells in basal culture medium comprising inorganic salts, amino acids, vitamins and at least one carbohydrate or metabolic product thereof and further comprising animal serum and IL-1 or IL-2.

In yet another aspect of the invention, there is provided a method of enhancing or extending immune or organ function in an individual suffering from a deficiency relating to a reduced number of functional macrophages in a tissue or organ. The method comprises administering a therapeutically effective amount of the replicating macrophages prepared in accordance with the present invention.

In still yet another aspect of the invention, there is provided a method of enhancing or extending immune or organ function in an individual suffering from a deficiency relating to a reduced number of functional macrophages in a tissue or organ. The method comprises administering a therapeutically effective amount of replicating macrophages prepared in accordance with the methods of the invention.

### BRIEF DESCRIPTION OF THE FIGURES

FIG. 1 is a graph showing growth in vitro of three human Kupffer cell isolates obtained from different individuals infected with HCV (the isolates initially were not HCV infected). Kupffer cell isolates cell growth with and without HCV infection is compared.

FIG. 2 is a graph showing extended growth in vitro of human Kupffer cells isolates.

### DETAILED DESCRIPTION OF THE INVENTION

The present invention makes possible the replication of macrophages by culture in vitro. In accordance with the methods of the invention, a source of macrophages isolated from an animal can be induced to undergo cell division, resulting in an expanded number of macrophages over time. In a preferred embodiment, the method can be used to replicate a population of Kupffer cells isolated from liver tissue such as from a liver biopsy.

An expanded source of macrophages prepared in accordance with the methods of the invention can be used to treat an individual suffering from a deficiency relating to a reduced number of functional macrophages in a tissue or organ. For example, in the case where an individual has liver disease, the methods of the invention can be used to prepare a non-infected source of Kupffer cells obtained from small sample of a failing liver. An expanded source of macrophages also can be used to for testing therapeutic agents for toxicity and efficacy, or for studying the life cycle of an agent that can infect a particular type of macrophage. For example, the life cycle of HCV in liver infection can be more easily evaluated using Kupffer cells prepared in accordance with the methods of the invention. A source of replicating macrophages also may be used as target cells for immunological assays or to process antigen during preparation of a vaccine.

The term “macrophage” are used herein is intended to include any of a variety of known members of the mononuclear phagocytic system which are present in all organs and tissues of the body as well as antigen presenting dendritic cells. Thus, macrophages include blood monocytes and phagocytic cells in various organs and tissues such as microglia of the nervous system, Kupffer cells of the liver, alveolar macrophages of the lungs, and the like. Dendritic cells are found throughout the body including follicular dendritic cells in lymph nodes and spleen and Langerhan's cells of the skin. Although not bound by any theory, macrophages are believed to derive from a bone marrow stem cell that first differentiates into a monocyte, which then leaves the marrow and enters the circulation. Monocytes then settle in tissues where they differentiate further into a macrophage (also known as histiocytes).

Macrophages useful for replicating may be obtained from blood or from a suitable tissue of an animal. In a preferred embodiment, the macrophages are obtained from a tissue source other than a tumor. In another embodiment, the macrophages are obtained from a non-embryonic animal (i.e. an animal that is not an embryo). As used herein, the terms animals includes all mammals, avians, reptiles, and the like. Humans are a preferred animal for providing and initial starting source of macrophages.

Macrophages suitable for use in the methods of the invention can be isolated from blood or from tissues by any of a variety of methods well known to those in the art. For example, blood monocytes may be isolated by centrifugation on Ficoll-Hypaque, while tissue macrophages may be isolated from tissue by macerating the tissue on a steel or nylon mesh followed by density gradient centrifugation (e.g. using Percol). Adherence to uncoated plastic surfaces such as the plastic surface of a tissue culture flask or tissue culture well plate may also be used to select for macrophages that can adhere to plastic, a feature common to most macrophages. Macrophages may be separated from other cells in culture by differential trypsinization.

In accordance with the methods of the present invention, isolated macrophages are cultured in vitro in a macrophage culture medium (“MCG”) comprising a basal culture medium and comprising an animal serum such as fetal calf serum (between about 5% to 25%) and IL-1 or IL-2 (between about 10 -200 U/ml). Under these conditions, at least some of the macrophages will undergo cell division during culture but are non-transformed. For optimal growth the MCG may include dimethyl sulphoxide (about 0.5 to 2%), hydrocortisone (between about 10⁻⁵ to 10⁻⁷ M) heparin (between about 20 to 60 mg/ml), Aminochrome II basal medium (about 5%; BioWhittaker) and endothelial cell growth supplement (“ECGS”) (between about 20 to 50 μg/ml). Preferably, the MCG contains little to no low-density lipoprotein. One skilled in the art would appreciate that the MCG can include other well-known culture medium supplements without diminishing the capacity of the medium to support macrophage growth.

A suitable basal medium comprises inorganic salts, amino acids, vitamins and at least one carbohydrate or metabolic product thereof. A preferred carbohydrate is glucose and a preferred metabolic product is pyruvate. The basal medium preferably is low in calcium, comprising a concentration of about 0.4 mM or less. Examples of such media include, for example, Ham's F10, Ham's F12, RPMI 1640, and the like. RPMI 1640 is a preferred basal media for replicating macrophages.

In a preferred embodiment, the present methods can be used prepare a culture of replicating Kupffer cells (“KC's”). These cells line the hepatic sinusoids and actively participate in the maintenance of normal liver functions. KCs exhibit vigorous phagocytosis, and produce many kinds of soluble mediators e.g., prostanoids, oxygen radicals, proteases, and cytokines. Activated KCs are the source of prostaglandins (PGE) when activated by ethanol, obstructive jaundice results due to the impaired phagocytic function of KCs. Chemokines (IL-12, IL-18 etc.) produced by KCs are also implicated in the pathogenesis of alcoholic liver disease. KCs are highly responsive to the effects of bacterial stimuli (endotoxin, for example lipopolysaccharide, and superantigens).

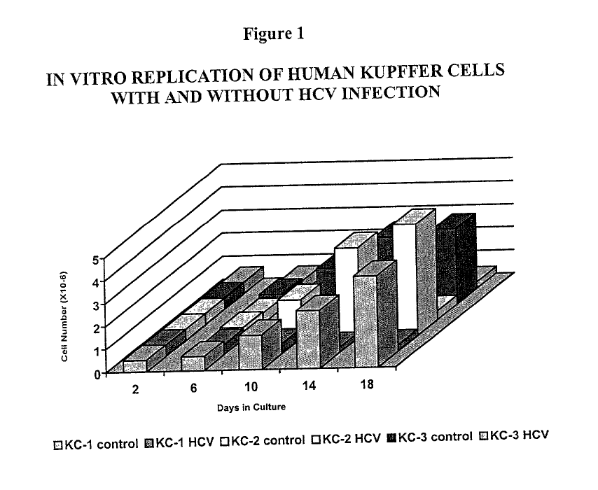

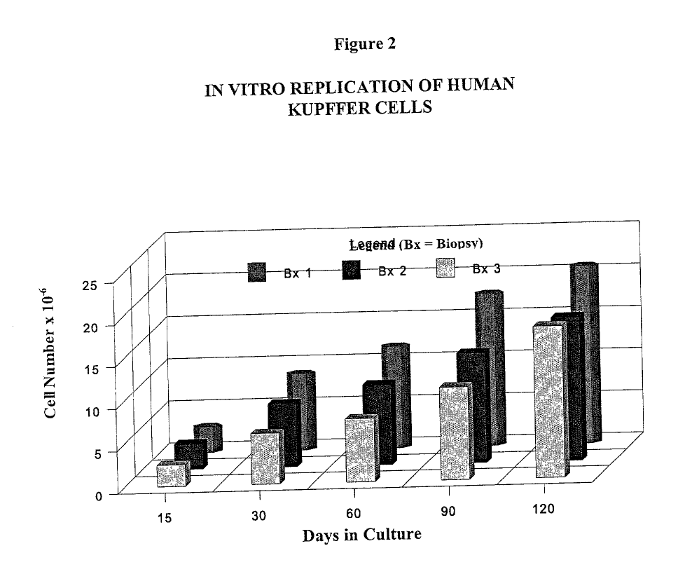

KCs can be reproducibly replicated (>95% efficiency) by in vitro culture from different patients using the methods disclosed herein. Replication is robust and can be maintained for extended periods of time, resulting in significant KC accumulation over time. As seen in FIG. 1, about >3-4×1⁶/ml live KC were obtained at 18 days culture while approximately 2×1⁷/ml live KC cells were obtained by approximately 120 days (FIG. 2). Morphologically, growing KC form monolayer colonies of stelate or cuboidal cells and require 7 to 10 days go to >90% confluency. The results are, however, dependent upon the presence of living cells in the biopsy specimen arriving in sterile condition.

KC can be prepared from freshly obtained liver biopsies using the fine needle aspiration technique. Liver tissue also can be taken during surgery or necropsy; In the latter case, the liver sample must be taken soon after death before the Kupffer cells have begun to die. The liver biopsy or sample may be from an individual suffering from a liver disease including a viral infection such as caused by hepatitis C virus. As described in the examples, the infected Kupffer cells may be separated from non-infected cells using single cell cloning methods. The examples also describe a suitable culture medium that was found to sustain growth of the KC isolates for more than a month, and even more than four months.

Morphological and cytochemical characteristics of various replicating Kupffer cell isolates are summarized in Table 1.

TABLE 1
Morphological and Cytochemical Characteristics of
Kupffer Cell Isolates Obtained
From Infected Liver Biopsies.

| | | Cytochemistry | | |
|---|---|---|---|---|
| Isolate | | Non-specific | Acid | |
| Number | Morphology | Esterase | phosphatase | Sudan Black |
| 1 | Epithelial-like | Positive | Positive | Negative |
| 2 | Epithelial-like | Positive | Positive | Negative |
| 3 | Epithelial-like | Positive | Positive | Negative |
| 4 | Mixed | Positive | Positive | Negative |
| 5 | Mixed | Positive | Positive | Negative |
| 6 | Epithelial-like | Positive | Positive | Negative |
| 7 | Epithelial-like | Positive | Positive | Negative |
| 8 | Epithelial-like | Positive | Positive | Negative |

TGFβ = Transforming growth factor beta
TNFα = Tumor necrosis factor alpha
NT—Not Tested

The expression of surface markers and phagocytosis of various replicating Kupffer cell isolates are summarized in Table 2.

TABLE 2
Cell Surface Markers and Phagocytic Ability of
Kupffer Cell Isolates Obtained
From Infected Liver Biopsies.

Immuno Cytochemistry

| Isolate | | TGFβ | |
|---|---|---|---|
| Number | CD 68 | TNFα | Phagocytosis |
| 1 | Positive | Negative | Positive |
| 2 | Positive | Negative | Positive |
| 3 | Positive | Negative | Positive |
| 4 | NT | Negative | Positive |
| 5 | Positive | NT | Positive |
| 6 | Positive | Negative | Positive |
| 7 | Positive | Negative | Positive |
| 8 | Positive | Negative | Positive |

TGFβ = Transforming growth factor beta
TNFα = Tumor necrosis factor alpha
NT—Not Tested

Kupffer cells grown using the methods of the present invention can be infected by a variety of viruses viz., HIV-1, HHV-6, and HCV. Evaluation of disease processes associated with HCV infection e.g., cirrhosis and lymphoma is now possible with replicating KC. Replicating KC are also useful in assays, since they facilitate the evaluation of therapeutic strategies for liver disease. Growing KC isolates also will aid the development of vaccine and other related subjects.

The cultures containing replicating macrophages prepared in accordance with the methods of the present invention can be used to enhance or extend immune function or organ function in an individual suffering from a deficiency relating to a reduced number of functional macrophages in a tissue or organ. The method comprises administering a therapeutically effective amount of replicating macrophages. Any suitable route including ip, iv, im, subq, and the like, may be used to administer such cells.

In a preferred embodiment the replicating Kupffer cells can be administered to an individual suffering from a liver disease where the disease results at least in part to a loss of Kupffer cell function. Illustrative of specific diseases include infectious hepatitis such as hepatitis resulting from HCV infection. The in vitro cultured KC may be transplanted by infusion i.v. or administration i.p., or through the portal vein of the liver of the patient. Transplantation also may include injection directly into the liver. Infusion through the portal vein of the liver is preferred method to transplant the cells. This latter route is relatively simple, safe and is conducted using commonly available and inexpensive equipment.

Enhancement or extension of immune function or organ function can be determined for the particular circumstances. For example, enhancement of liver function may involve an increase in any of a variety of liver function measurements well known on the art (e.g., bilirubin, SGOT, etc.). Extension of liver function occurs when any measurement of liver function is increased following the administration of KC as compared to the predicted function in the absence of KC administration (with all other treatment the same).

Toxicity and therapeutic efficacy can be determined by standard pharmaceutical procedures in experimental animals. Animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Doses of cells that exhibit large therapeutic indices are preferred. The data obtained from animal studies can be used in formulating a range of dosages suitable for use in humans. The dosage of such mutants lies preferably within a range of cell concentrations that include the ED₅₀ with little or no toxicity. The dosage may vary within this range depending upon a variety of factors, e.g., the dosage form employed, the route of administration utilized, the condition of the subject, and the like.

A therapeutically effective dose can be estimated initially from animal assays by determining an IC₅₀. A dose can then be formulated in animal models to achieve an initial circulating cell concentration range that includes the IC₅₀ as determined in animals. Such information can be used to more accurately determine useful doses in humans. The exact formulation, route of administration and dosage can be chosen by the individual physician in view of the patient's condition.

The attending physician for patients treated with replicating macrophages would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Replicating Kupffer cells for transplantation to liver on can be of great benefit as a temporary or longer-term solution to liver disease. Liver transplant is the established therapy of choice for end stage acute and chronic liver diseases of various etiologies. Liver transplantation achieves a five-year survival on average. It is estimated that while there are approximately fifteen thousand patients waiting for liver transplant in these United States, only 4500 donor livers appropriate for transplant, becomes available every year. The surgical rescue by the traditional process for these patients is not only risky it is also extremely expensive. The average estimated cost of each transplant is $300,000²⁴. The ability to grow KC from an individual patient makes it possible to return these cultured cells to the individual.

Transplantation of liver disease patients with replicating Kupffer cells will extend the survival time for patients who are candidates for liver transplants but do not have a suitable organ donor available. Optimally, such candidates will receive an autolgous transplant providing short term relief that was expanded in accordance with the methods of the invention from a biopsy taken earlier from that same individual. Transplantation of KC prepared as described herein will seed the failing liver with the freshly cultured KCs taking up residence in available areas restoring varying levels of metabolic, synthetic and other liver functions. In short, the opportunity to transplant in vitro grown KCs from the impaired livers of patients will reduce the need for organ transplants and provide a painless, durable treatment for some patients. This procedure may be the only choice for the patients that do not qualify for whole liver transplantation due to their age, frailty and other causes.

Transplantation using autologous KC is preferred over allogeneic KC to avoid rejection based on tissue histocompatibility antigens (HLA) and the consequent immunological complications for mismatch. However, transplantation of non-autologous KC such as allogeneic or xenogeneic cells can be used for short-term enhancement or extension of liver function. Such cells also can be transplanted when the incompatibility is minimal or when transplantation is combined with an appropriate immunosuppressive (or tolerogenic) regimen (e.g., cyclosporin A, ant-CD3 antibody, and the like).

The following are non-limiting examples of the invention.

### EXAMPLES

### Example 1

Materials and General Methods

Sources of Material: Fresh Liver biopsies routinely obtained from Myron Tong Ph.D., MD, Gastro-enterologist and liver specialist at Huntington Memorial Hospital.

Growth Factors: Endothelial cell growth supplement (ECGS); basic fibroblast growth factor (bFGF); epidermal growth factor (EGF); platelet derived growth factor (PDGF); interleukin-1 (IL-1); nerve growth factor tumor necrosis factor alpha (TNFa); interferon (INF) (Amgen Biologicals, Thousand Oaks, Calif.); transforming growth factor β (TGF-β, R&D Systems, Inc., St. Louis, Mo.); interleukin-1, and interleukin-2 (IL-1, IL-2, Genzyme, Norwalk, Conn.); granulocyte-monocyte colony stimulating factor (GMCSF) (Genzyme, Boston, Mass.), Hydrocortisone (SIGMA, St. Louis, Mo.), and affinity chromatography material (Pharmacia, Upsalla, Sweden), were obtained as indicated.

Tissue Culture Media: Cells were grown in RPMI 1640 media supplemented with Nutridoma-hu (Boehringer Manheim,) and Glucagon (0.1% v/v; Eli Lilly, Indianapolis, Ind.).

Cytological Analysis: Non-specific esterase, acid phosphatase and Sudan Black staining was performed using previously published procedures. Salahuddin et al., Science 242:430-433 (1988)).

Immunocytochemistry was performed using monoclonal antibodies and sources as follows: anti-FVIIIR-Ag (Cappel, West Chester, Pa., 19380) and (DAKOPATTS, Santa Barbara, Calif., 93103); monoclonal anti-tissue plasminogen activator inhibitor (TPA I) antibodies, MC-1, -2, and -3 (kindly provided by Dr. B. Blomback, Karolinska Institute, Sweden); EN-4 and PAL-E (Sambio bv, 5400 Am. Uden, Holland); anti-fibronectin (Hybritech, San Diego, Calif., 92121); anti-vimentin, anti-desmin and anti-cytokeratin (Boehringer Mannheim, Indianapolis, Ind.) and anti-keratin (AEI and NAE-3; Hybritech) were diluted in PBS or 0.05 M Tris-HCL (pH=7.6) before use. For immunochemistry, rabbit anti-mouse immunoglobulin (APAAP-mouse; DAKOPATTS) was used as a second antibody, which was diluted 1:20 and incubated at 37° C. for 60 minutes. Fast red (Vector Laboratories, Inc., Burlington, Calif., 94010) was used as substrate.

Angiotensin Converting Enzyme (ACE) Assay was performed using ³H-benzoyl-phe-ala-pro as a substrate (Ventrex Laboratories, Inc., Portland, Me. 04103), according to the Ventrex manual. By extraction with Ventrex Scintillation cocktail #2, unreacted substrate was separated from ³H-benzoyl-phe in organic phase and was counted in liquid scintillation counter. The activity of all cell lines was also tested by the addition of specific inhibitor for ACE, SQ 20,881 (Peninsula Laboratories, Belmont, Calif., 94002). 90-95% of this activity was inhibited with 5×10⁻⁶M of this inhibitor.

FITC-conjugated Ulex Europaeus-I Agglutimin (UEA-I) (Vector Laboratories, Inc.) was incubated with acetone-fixed cells at 37° C. for 1 hour. The specificity of UEA-I, the lectin was determined by preincubating with 0.2M L-fructose (Sigma), at room temperature for 30 minutes. No binding was observed under this condition.

Acetylated Low Density Lipoprotein (Ac-LDL) uptake was determined using 1,1′-dioctadecyl-1-1,3,3′,3′-tetramethyl-indo-carbocyamine perchlorate (Dil-AcLDL)(Biomedical Technologies, Inc., Cambridge Mass., 02141) as probe. 10 μg/ml of Dil-Ac-LDL was incubated with live cells on the slides, at 37° C. for 4 hours. The cells were washed with probe-free media 4-5× and kept in media for observation. Uptake was visualized under fluorescence microscope with standard rhodamine excitation emission filter (−=Negative, +=weak positive, ++=moderate positive, +++=strong positive ).

DNA Synthesis Assay: DNA synthesis was determined by culturing the cells with ³H-Thymidine as is standard in the art. The KC isolates used in the assay were at day 21 post liver isolation. The normal endothelial cells and foreskin fibroblasts were cultured in media consisting of RPMI-1640, fetal bovine serum 20% supplemented with specific factors as described in Nakamura S and Salahuddin SZ, Science 242: 426-430,1988. KC isolates were cultured as described above. Cells were harvested between 4 hours and 18 hours culture depending on the stimulatory agent used. A stimulation index was calculated by dividing radioactivity incorporated into cells with the stimulatory agent versus cells in culture medium alone.

Cultured Cells: Human umbilical vein endothelial cells (HUV-E) were prepared as described in Nakamura et al, Science 1988. Bovine capillary endothelial cells (BCE) were a gift from Dr. Peter Biberfeld of Karolinska Institute, Stockholm, Sweden. Normal human foreskin fibroblasts (HSF) were obtained from the American Type Tissue Culture Collection (ATCC), Rockville, Md. (CRL-1635).

### Example 2

Preparation of Replicating Human Kupffer Cell Cultures

Biopsies of human liver were obtained by routine fine needle aspiration or by open surgical biopsy from HCV infected individuals. The biopsy was digested with trypsin-EDTA (0.05% w/v trypsin and 0.02% w/v EDTA; ICN Biomedical, Aurora Ohio) for about one hour at 37° C. with gentle agitation to release cells from the tissue. RPMI-1640 medium without serum or other additive was then added (usually 5 to 10 ml depending on the volume of the trypsin-EDTA solution) and the tissue was subjected to gentle pipetting. The cell suspension was layered over Ficoll-Hypaque and then subjected to centrifugation at 700×g for 15 minutes. Banded cells were removed and rinsed by centrifugation in a generous amount of RPMI-1640. Non-HCV infected KC were isolated from HCV infected cells by limiting dilution in 96 well plates. Briefly, banded cells were plated overnight in 96 well plates and non-adherent cells removed by gentle washing. The wells were inspected during subsequent culture to identify wells having a showing KC replication from a single initial adherent cell.

A cell culture medium for growing macrophages (Macrophage Cell Growth medium or “MCG medium”) was prepared in advance. MCG medium comprised RPMI-1640 supplemented with fetal calf serum (20% v/v), HepatoZYME-SFM (5% v/v; GibcoBRL, catalog number 17705-021), Aminochrome-II basal medium (5% v/v; Bio-Whittaker), L-Glutamine (2 mM), hydrocortisone (water soluble; 10⁻⁶ M) sodium pyruvate (1 mM) and dimethyl sulphoxide (1%), heparin (45 mg/ml), endothelial cell growth supplement (“ECGS”) (30 μg/ml; from Dr. Parkash Gill, USC; also available from commercial sources such as Upstate Biotechnology, Lake Placid, N.Y.; catalog no. 02-101), and recombinant interleukin-2 (“IL-2”) (100 U/ml) (purchased from Collaborative Research Inc., formerly of Waltham Mass.). After addition of supplements, MCG medium was filtered through a 0.45 m filter (Fisher Scientific). MCG medium is designed to be low in calcium (about 0.4 mM) and free or nearly free of low-density lipopolysaccharide.

The banded and rinsed cells were suspended in freshly prepared MCG medium and placed in a gelatinized tissue culture flask of appropriate size. The cell cultures were maintained at 37° C. in an atmosphere containing ˜5% CO₂. On freshly initiated cultures the medium was changed every three days. Upon confluence, cells were removed from by trypsinization, washed by centrifugation and subcultured (i.e. passaged) by seeding new flasks at a density of 10⁶/T-75 cell culture flasks.

Morphological analysis of eight different KC isolates after three weeks of growth showed epithelial like morphology except for isolates 4 and 5, which exhibited a mixed morphology (Table 1).

KC showed different cytochemical characteristics from that of H-UVE cells. ATPase and angiotensin converting enzyme (ACE), which are known to be present in vascular endothelium, were detectable in H-UVE only. The KC isolates were positive for naphthyl acetate esterase (non-specific estrase), acid phosphatase, but negative for alkaline phosphatase and Sudan Black (Table 1).

The pattern of reactivities seen in the KC isolates, i.e., negative results for factor VIII R-Ag, EN-4, PAL-E, ATPase, alkaline phosphatase and ACE and positive results for TPA-I, 5′ nucleotidase, UEA-I and Ac-LDL as well as the characteristic of phagocytosis are consistent with the cells having a macrophage origin (Table 2). The factor VIII related antigen (“factor VIII: Ag”) described by Jaffe et al. (Proc. Nat'l Acad. Sci, USA, 71, 1906 (1974)) and commonly found in endothelial cells of vascular origin, was detectable in normal human vascular endothelial cell line (H-UVE) but not in the KC isolates. The EN-4 monoclonal antibody described by Cui et al (Immunology, 49, 183 (1983)), which detects a plasma membrane antigen of vascular and lymphatic endothelial cells, showed no detectable binding with the KC isolates or with H-UVE (Table 2). The PAL-E monoclonal antibody, which recognizes an antigen selected with pinocytic vesicles in cells of the vasculature but not lymphatics, bound to H-UVE cells only. Both H-UVE and the KC isolates reacted with antibody against an inhibitor of plasminogen activator, TPAI, which is secreted by endothelial cells. Both the KC isolates and the H-UVE cells failed to react with antibodies to cytokeratin, expressed by ectodermal cells or to vimentin or cytokeratin, expressed in skeletal, visceral and certain vascular smooth muscle cells. The KC isolates also expressed CD68 but were negative for expression of UEA-1 (Table 2).

### Example 3

Stimulating Growth of Kupffer Cell Culture

Various growth factors were tested for their ability to stimulate growth of the human Kupffer cell isolates (KC-3) and other cell types. KC isolates used in this experiment were grown in culture for 21 days with MCG medium. Table 3 shows that ECGS,

bFGF, EGF and PDGF stimulated DNA synthesis of normal endothelial cells (HUVE and BCE and to a much lesser extent foreskin fibroblasts, but had little to no effect on the KC isolates. In contrast, Stimulocyte stimulated DNA synthesis in KCs to a greater extent that for the normal endothelial cells. IL-I, and IL-2 weakly simulated DNA synthesis of KC isolates and normal endothelial cells, although to a lesser extent than Stimulocyte.

TABLE 3
STIMULATION OF DNA SYNTHESIS BY SELECTED
GROWTH FACTORS
Stimulation Index of Cultured Target cells

| Factors | HC¹ | KC-1⁹ | KC-2 | KC-3 | HUVE¹⁰ | BCE¹² | HFS¹³ |
|---|---|---|---|---|---|---|---|
| Stimu- | + | 5.2 | 4.8 | 4.6 | 2.8 | 1.8 | 0.8 |
| locyte² | − | 4.0 | 3.8 | 3.2 | 3.2 | 1.4 | 1.1 |
| ECGS³ | + | 1.1 | 0.6 | 1.1 | 12.4 | 6.6 | 2.0 |
| | − | 0.9 | 0.8 | 1.2 | 20.8 | 8.6 | 2.2 |
| BFGF⁴ | + | 0.5 | 0.5 | 1.2 | 15.4 | 2.3 | 1.9 |
| | − | 0.8 | 0.6 | 1.2 | 35.4 | 6.2 | 2.2 |
| EGF⁵ | + | 0.9 | 0.9 | 0.6 | 10.0 | 2.9 | 1.1 |
| | − | 1.2 | 1.1 | 1.2 | 25.4 | 6.8 | 2.8 |
| PDGF⁵ | + | 1.1 | 1.2 | 1.1 | 20.2 | 1.8 | 1.5 |
| | − | 1.2 | 1.4 | 1.4 | 10.0 | 4.4 | 1.4 |
| IL-1⁶ | + | 3.1 | 2.8 | 2.8 | 2.0 | 1.5 | 0.8 |
| | − | 3.0 | 2.4 | 2.8 | 2.8 | 2.0 | 0.8 |
| IL-2⁷ | + | 2.1 | 2.9 | 2.8 | 3.2 | 2.8 | 2.0 |
| | − | 1.8 | 2.4 | 2.8 | 2.8 | 2.0 | 2.0 |
| TNFα⁸ | + | 0.8 | 0.9 | 1.2 | 1.0 | 1.2 | 0.8 |
| | − | | | | | | |

Legend to Table 3:
¹HC = Hydrocortisone, lot # 39F-8878, Sigma Chemical, Mo.
²Stimulocyte Lot # 91-0467, Collaborative Research
³Endothelial Cell Growth Supplement, Gift from Dr. Parkash Gill, University of Southern California Medical School, Los Angeles, CA
⁴bFGF = Fibroblast Growth Factor β, Lot # 14765800, Boehringer Manheim
⁵PDGF = Platelet Derived Growth Factor, Gift from Virotech Laboratories, Rockville, Md
⁶IL-1 = Interleukin-1, Lot # B9158, Genzyme, Norwalk, CT
⁷IL-2 = Natural product, lot # 019, Genzyme Inc., Norwalk, CT
⁸TNFα = Tumor Necrosis Factor α, Lot # 3056-63, Genentech, So San Francisco, CA
⁹KC = Kupffer's Cells # 1, 2, 3
¹⁰HUV-E = Human Umbilical Vein Endothelial Cells
¹¹BCE = Bovine Capillary Endothelial Cells
¹²HSF = Human Foreskin Fibroblast Cells

The growth rate of day 2 KC isolates was compared to that of H-UVE, BCE and HSF cells grown under optimal conditions. The results in Table 4 (shown graphically in FIG. 1) show growth of all three KC isolates at slightly lower rates that for the other cell lines.

TABLE 4
In Vitro Replication of Kupffer Cell Isolates and
Other Cell Types With or Without
Infection with HCV.
Cell number × 10e6/ml

| | | Days | | | | |
|---|---|---|---|---|---|---|
| Celltype | | 2 | 6 | 10 | 14 | 18 |
| KC-1 | control | 0.50 | 0.62 | 1.50 | 2.53 | 3.99 |
| | HCV | 0.50 | 0.45 | 0.43 | 0.24 | 0.29 |
| KC-2 | control | 0.50 | 0.53 | 1.68 | 3.89 | 4.87 |
| | HCV | 0.50 | 0.45 | 0.42 | 0.39 | 0.35 |
| KC-3 | control | 0.50 | 0.62 | 1.69 | 3.00 | 3.29 |
| | HCV | 0.50 | 0.49 | 0.43 | 0.29 | 0.30 |
| H-UVE** | control | 0.50 | 0.62 | 2.10 | 3.99 | 4.90 |
| | HCV | 0.50 | 0.62 | 2.00 | 3.65 | 3.76 |
| BCE** | control | 0.50 | 0.57 | 2.38 | 3.97 | 5.11 |
| | HCV | 0.50 | 0.57 | 2.13 | 3.65 | 4.88 |
| HSF** | control | 0.50 | 0.64 | 2.48 | 4.68 | 5.34 |
| | HCV | 0.50 | 0.64 | 2.33 | 4.33 | 4.52 |

**Not infectable by human hepatitis C virus
**These cells were cultured with appropriate supplements for optimum growth condition
Cell numbers have been reduced to two decimal points.

In contrast, prior HCV infection of KC isolates completely inhibited growth but had no affect on growth of the other cells, the latter cells not supporting infection by HCV). FIG. 2 shows that KC isolates KC-1, KC-2 and KC-3 continued to replicate for at least 120 days in culture, showing a 15-20 fold increase in cell number over this time period.

KC cell lines will be deposited with the American Type Culture Collection (Rockville Md.) ATCC Patent Culture Depository under the terms and conditions of the Budapest Treaty and in accordance with U.S. law under 35 U.S.C. § 122 and 37 C.F.R. §§ 1.806-1.808.

The invention thus has been disclosed broadly and illustrated in reference to representative embodiments described above. Those skilled in the art will recognize that various modifications can be made to the present invention without departing from the spirit and scope thereof. All publications, patent applications, and issued patents, are herein incorporated by reference to the same extent as if each individual publication, patent application or issued patent were specifically and individually indicated to be incorporated by reference in its entirety. Definitions that are contained in text incorporated by reference are excluded to the extent that they contradict definitions in this disclosure.

That which is claimed is:

1. A composition comprising a culture of replicating macrophages wherein at least some of the macrophages have undergone cell division during culture in vitro.

2. The composition of claim 1 wherein said replicating macrophages have undergone cell division during culture in vitro for at least one month.

3. The composition of claim 1 wherein said replicating macrophages have undergone cell division during culture in vitro for at least four months.

4. The composition of claim 1 wherein said replicating macrophages are phagocytic.

5. The composition of claim 1 wherein said replicating macrophages stain positive for non-specific esterase and acid phosphatase.

6. The composition of claim 1 wherein said replicating macrophages express CD68

7. The composition of claim 1 wherein said replicating macrophages do not express TGFβ.

8. The composition of claim 1 wherein said replicating macrophages are Kupffer cells.

9. The composition of claim 1 wherein said replicating macrophages are human macrophages.

10. The composition of claim 1 wherein said replicating macrophages are non-transformed.

11. The composition of claim 1 wherein said replicating macrophages are from a tissue source other than a tumor.

12. The composition of claim 1 wherein said replicating macrophages are from a non-embryonic animal.

13. A method of culturing macrophages in vitro such that at least some of the macrophages have undergone cell division during culture, said method comprising growing the cells in basal culture medium comprising inorganic salts, amino acids, vitamins, at least one carbohydrate or metabolic product thereof and further comprising animal serum and IL-1 or IL-2.

14. The method of claim 13 wherein said culture medium further comprises dimethyl sulphoxide and hydrocortisone.

15. The method of claim 13 wherein said culture medium further comprises heparin.

16. The method of claim 13 wherein said animal serum is fetal calf serum.

17. The method of claim 13 wherein said culture medium comprises IL-2.

18. The method of claim 13 wherein said macrophages are human macrophages.

19. The method of claim 13 wherein said macrophages are Kupffer cells.

20. The method of claim 19 wherein said Kupffer cells are isolated from human liver by needle biopsy.

21. The method of claim 19 wherein said Kupffer cells are isolated from liver of an individual suffering from a liver viral infection.

22. The method of claim 21 wherein said Kupffer cells are not virally infected.

23. The method of claim 13 wherein said macrophages continue to replicate for at least one month.

24. The method of claim 13 wherein said macrophages continue to replicate for at least four months.

25. The method of claim 13 wherein said replicating macrophages are non-transformed.

26. The method of claim 13 wherein said macrophages are from a tissue source other than a tumor.

27. The method of claim 13 wherein said macrophages are from a non-embryonic animal.

28. A composition comprising replicating Kupffer cells prepared by the method of claim 13.

29. A method of enhancing or extending immune or organ function in an individual suffering from a deficiency relating to a reduced number of functional macrophages in a tissue or organ, said method comprising administering a therapeutically effective amount of the macrophages of claim 1.

30. The method of claim 29 wherein said individual is a human.

31. The method of claim 29 wherein said organ is liver.

32. The method of claim 29 wherein said macrophages are Kupffer cells.

33. The method of claim 29 wherein said deficiency is due to hepatitis C virus infection.

34. A method of enhancing or extending immune or organ function in an individual suffering from a deficiency relating to a reduced number of functional macrophages in a tissue or organ, said method comprising administering a therapeutically effective amount of the macrophages of claim 8.

35. The method of claim 34 wherein said individual is a human.

36. The method of claim 34 wherein said organ is liver.

37. The method of claim 34 wherein said macrophages are Kupffer cells.

38. The method of claim 34 wherein said deficiency is due to hepatitis C virus infection.
